(19) United States
(12) Patent Application Publication
(10) Pub. No.: US 2018/0120232 A1
(43) Pub. Date: May 3, 2018

(54) PHARMACEUTICAL DETECTION

(71) Applicant: UNIVERSITY COURT OF THE UNIVERSITY OF ST ANDREWS, St Andrews, Fife (GB)

(72) Inventors: Derek CRAIG, St Andrews, Fife (GB); Kishan DHOLAKIA, St Andrews, Fife (GB); Michael MAZILU, St Andrews, Fife (GB)

(21) Appl. No.: 15/561,878

(22) PCT Filed: Mar. 1, 2016
(86) PCT No.: PCT/GB2016/050531

§ 371 Date: Sep. 26, 2017

(30) Foreign Application Priority Data
Mar. 27, 2015 (GB) 1505297.0

(51) Int. Cl. G01N 21/65; G01N 33/15

(52) U.S. Cl. CPC G01N 21/65; G01N 2021/6419; G01N 33/15

(57) ABSTRACT

A method for detecting or identifying an analyte, the method comprising: applying an analyte in fluid, for example a drug, to a paper microfluidic device; exciting Raman scattering in the analyte in the paper microfluidic device at a multiple different wavelengths; capturing a signal at each wavelength; and analyzing the captured signal at each wavelength to identify a Raman signal associated with the analyte.

### CROSS-REFERENCE TO RELATED APPLICATION(S)

This application is a national stage application (filed under 35 § U.S.C. 371) of PCT/GB2016/050531, filed Mar. 1, 2016 of the same title, which, in turn claims priority to Great Britain Application No. 1505297.0, filed Mar. 27, 2015 of the same title; the contents of each of which are hereby incorporated by reference.

### FIELD OF THE INVENTION

The present invention relates to detection of pharmaceutical products using Raman spectroscopy and paper based microfluidics.

### BACKGROUND OF THE INVENTION

Recently, paper based sensing has emerged in the field of point of care testing with applications in the area of biosensing, environmental monitoring and food quality control. Paper provides a means by which microfluidic devices can be fabricated in a very low-cost, simple and reproducible manner. Patterning of paper using techniques such as ink-jet and wax printing produces defined hydrophilic channels in the paper structure. These control the flow of liquid through the sensor. Due to the inherent wicking capability of paper, the passive transport of liquid through pre-defined channels is possible and a vast range of chemicals have been shown to be compatible with the paper substrate.

Paper microfluidics has emerged as a promising complementary technique to current microfluidic technologies with the key advantage of not requiring significant external instrumentation (e.g. microfluidic pumps) to function. Paper microfluidics has the promise to realize a lab on a chip device due to the approach being fast, simple to implement as well as offering ease of transport and disposal.

Although there are significant promising advantages of paper microfluidics, a number of limitations, such as poor accuracy and sensitivity, constrain their applications. Currently, a number of detection techniques are being explored to overcome such disadvantages. These include colorimetric, electrochemical and fluorescent detection technologies.

Optical approaches such as Raman spectroscopy confer the possibility of label free detection of analytes on paper microfluidic devices. However, this technique in its native form has so far been obviated in favour of surface enhanced Raman scattering (SERS). To date, notable uses of SERS include the quantitative detection of narcotics, such as cocaine and heroin, and the development of ELISA type formats to detect antigen-antibody interactions. These examples have been successful in achieving the detection of analytes down to nanomolar concentrations. The fabrication of paper based SERS substrates relies on deposition of an enhancement material, such as nanoparticles or nanorods, onto a paper substrate. However, there are challenges to employing this technique, such as, difficulty in achieving a uniform covering of the paper substrate with the enhancing material and the loss of key functionalities such as separation and pre-concentration on the paper device. Also, the reproducibility achieved using SERS can be highly variable. Attempts to control this enhancement have led to different techniques being introduced to improve the reproducibility and fabrication of the SERS substrates.

Whilst Raman spectroscopy is a powerful analytical technique, the signal obtained from Raman scattering is typically weak due to only 1 in 10⁶ photons being Raman scattered. Hence, it can be easily obscured due to auto-fluorescence from the substrate or the sample being analyzed. Numerous techniques have been employed to suppress background fluorescence including time resolved Raman spectroscopy and shifted excitation Raman difference spectroscopy (SERDS). Another option is wavelength modulated Raman spectroscopy. This involves recording a series of Raman spectra, which are slightly shifted in excitation wavelength (<1 nm) with respect to one another. Using multivariate, principal components analysis (PCA) the modulated Raman information can be recovered and the fluorescent signal eliminated from the Raman signal.

### SUMMARY OF THE INVENTION

According to the present invention, there is provided a method for detecting or identifying an analyte, the method comprising: applying an analyte in fluid, for example a drug, to a paper microfluidic device; exciting Raman scattering in the analyte in the paper microfluidic device at a series of different wavelengths, capturing a signal at each wavelength; and analyzing the captured signal at each wavelength to identify a Raman signal associated with the analyte.

By using wavelength modulated Raman spectroscopy, the drawbacks of paper microfluidics, in particular relating to fluorescence of the paper, can be overcome. Using wavelength modulated Raman spectroscopy the inherent background fluorescence from the paper substrate can be eliminated. The approach is inherently simple and powerful, and can yield quantitative information.

Raman spectroscopy is based on the inelastic scattering of light from a sample. The resulting spectrum of the scattered photons reflects a shift in frequency characteristic of specific vibrational modes of the analyte being interrogated. As a result of this, a fingerprint spectrum is obtained from which individual analytes can be detected. Multiple analytes can be distinguished simultaneously.

The method may involve analyzing the captured signal using a principal component analysis (PCA) to recover the modulated Raman information.

The method may involve varying the excitation wavelength by a predetermined amount, for example 1 nm, so that the series of different wavelengths comprises a series of wavelengths separated by said predetermined amount, e.g. 1 nm.

The method may involve using the Raman signal to detect or identify the analyte.

The method may involve applying a known analyte to the paper microfluidic device and using the Raman signal as a fingerprint for that analyte.

The method may comprise creating a library of at least one fingerprint for at least one known analyte. Preferably, the known analyte is a known or authenticated drug.

The method may further involve identifying a Raman signal associated with an unknown analyte using the method of the invention and comparing it with the at least one fingerprint for the at least one known analyte.

According to another aspect of the invention, there is provided a system adapted to detect or identify an analyte, the system comprising: a sample holder for holding a paper microfluidic device to which an analyte has been applied; an excitation source for exciting Raman scattering in the analyte in paper microfluidic at a series of different wavelengths, a detector for capturing a signal from the device at each wavelength; and an analyzer for analyzing the captured signal at each wavelength to identify a Raman signal, and use the Raman signal to detect or identify the analyte.

According to another aspect of the invention, there is provided a method for detecting counterfeit drugs, the method comprising: applying drug in fluid to a paper microfluidic device; exciting Raman scattering in the drug in the paper microfluidic device at a multiple different wavelengths; capturing a signal at each wavelength; analyzing the captured signal at each wavelength to identify a Raman signal associated with the drug, and comparing the Raman signal associated with the drug with a stored Raman signal associated with a known, authenticated drug. In the event that the Raman signal associated with the drug, and the stored Raman signal associated with a known, authenticated drug are substantially the same, the drug is identified as being authentic. Otherwise, the drug is identified as being counterfeit.

According to yet another aspect of the invention, there is provided a system for detecting counterfeit drugs using a paper microfluidic device, the system being adapted to: excite Raman scattering in a drug in fluid form applied to the paper microfluidic device at a multiple different wavelengths; capture a signal at each wavelength; analyze the captured signal at each wavelength to identify a Raman signal associated with the drug, and compare the Raman signal associated with the drug with a stored Raman signal associated with a known, authenticated drug.

### BRIEF DESCRIPTION OF THE DRAWINGS

Various aspects of the invention will now be described by way of example only, and with reference to the accompanying drawings, of which:

FIG. 1 a schematic diagram of a process for making a paper microfluidic;

FIG. 2 is a block diagram of a system for detecting and/or identifying analytes using a paper microfluidic device and Raman spectroscopy;

FIG. 3 is a bar chart showing the average signal to noise ratio as a function of the number of modulation cycles, for four different wavelength modulations;

FIG. 4 is a table of measured signal to noise ratio as a function of the number of modulation cycles (5-30) and modulation amplitude (Δλ), at three different exposure times: (a) 3 s exposure time, (b) 4 s exposure time and (c) 5 s exposure time;

FIG. 5(a) shows a standard Raman spectrum of a paper microfluidic device;

FIG. 5(b) shows a wavelength modulated Raman spectroscopy spectra for a paper microfluidic device;

FIG. 5(c) shows a wavelength modulated Raman spectroscopy spectra for a paper microfluidic device and paracetamol;

FIG. 5(d) shows a wavelength modulated Raman spectroscopy spectra for a paper microfluidic device and ibuprofen;

FIG. 6(a) is a principal component analysis of a wavelength modulated Raman spectroscopy study of paper microfluidics device (green), as well as paper and paracetamol (blue) and paper and ibuprofen (red);

FIG. 6(b) shows a standard Raman study of a paper microfluidics device (green), as well as paper and paracetamol (blue) and paper and ibuprofen (red);

FIG. 7(a) shows a PCA scatter plot of PC2 vs. PC1 for an analysis of paper and paracetamol (blue) vs. paper device only (green);

FIG. 7(b) shows a PCA scatter plot of PC2 vs. PC1 for an analysis of paper and ibuprofen (red) vs. paper device only;

FIG. 8 shows a PCA scatter plot, PC2 vs. PC1, for analysis of varied concentrations of paracetamol on individual paper devices; and

FIG. 9 shows a PCA scatter plot, PC2 vs. PC1, for analysis of varied concentrations of ibuprofen on individual paper devices.

### DETAILED DESCRIPTION OF THE INVENTION

The present invention combines paper microfluidics and wavelength modulated Raman spectroscopy for sensitive detection of analytes. Paper microfluidics is a low cost, easy to fabricate and portable approach for point of care testing. Combining Raman spectroscopy with paper microfluidics was previously an unmet challenge in the absence of using surface enhanced mechanisms. Using wavelength modulated Raman spectroscopy allows the background fluorescence of the paper to be suppressed, and so enables the implementation of this technique for pharmaceutical analysis. Using paper microfluidics and wavelength modulated Raman spectroscopy, it is possible to discriminate between analytes, for example paracetamol and ibuprofen, whilst, also being able to detect the presence of each analyte quantitatively at nanomolar concentrations.

Wavelength modulated Raman spectroscopy involves capturing Raman spectra at multiple different wavelengths, so that an individual spectrum is available for each wavelength. Background fluorescence is typically independent of wavelength, but the Raman signal is sensitive to wavelength. By using the individual spectra at each wavelength signal variation between different spectra can be attributed to the Raman signal, whereas constant non-varying parts of the different spectra can be attributed to background fluorescence. Hence, by identifying variations between the different spectra, the Raman signal can be distinguished.

A particularly useful technique for identifying the varying, wavelength dependent Raman signal is principal components analysis (PCA). This is a statistical technique used to change and reduce the representation of a multidimensional data set. A new representation or coordinate system is constructed such that the variance of the data sets is biggest for the first coordinate component of the new representation. This is then called the first principal component. The second biggest variation lies on the second coordinate of the new representation, and so on. If the wavelength modulated spectra are fed into a PCA routine, the resulting first principal component describes the variation observed in the spectra. Because of the wavelength modulation, this variation is the moving Raman spectrum only, as the fluorescence remains steady. Thus, the PCA routine outputs a spectrum, or principal component that is an effective differential Raman spectrum of the sample.

An example of wavelength modulated Raman spectroscopy is described in the paper “Optimal algorithm for fluorescence suppression of modulated Raman spectroscopy”, Mazilu M et al (2010), Optics Express 18: 11382-11395, the contents of which are incorporated herein by reference.

When applied to paper microfluidics, the steps involved in the wavelength modulated Raman spectroscopy can be summarized as follows. Firstly, the analyte of interest is applied in fluid form to the paper sample, and multiple spectra from each paper sample are captured. Typically, ten spectra are used, each at predetermined wavelengths, separated for example by 1 nm. Ideally, the spectra are normalized with the total spectral intensity calculated by integrating over all spectral data (using Matlab 2014b). Normalization allows for compensation for any power fluctuation in the laser during wavelength modulation. Once this is done, principal component analysis (PCA) is used to analyze the normalized spectra collected, with each excitation wavelength step as a parameter. This produces a modulated Raman spectrum with essentially all fluorescence background suppressed. This modulated Raman spectrum is defined by the first principal component of the PCA. Within this representation, all standard Raman peaks are indicated by the zero crossing points and the modulated Raman spectrum is similar to a differential spectrum.

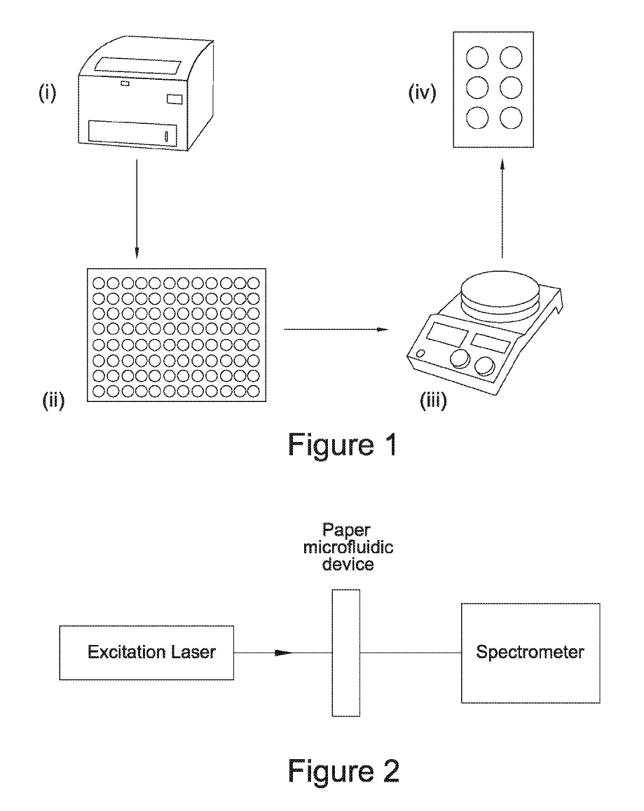

FIG. 1 shows the steps for preparation of a paper microfluidic device for use in wavelength modulated Raman spectroscopy. In a first step (i), the device was designed using Microsoft Powerpoint. The device was printed, step (ii) using a Xerox 8850DN solid wax printer onto an A4 sheet of Whatman No. 1 filter paper. The sheet of filter paper was then heated to 150° C. for two minutes to re-distribute the wax, see step (iii), to disperse the wax through both sides of the paper to create the 3D channels desired. After heating, the devices were cut to size (length: 2.5 cm, width 1.5 cm) and allowed to cool prior to being used.

In tests, each of the pharmaceuticals was diluted to the required concentration using purified MilliQ water. Of the resulting solution 10 mL were deposited into a 50 mL plastic sampling tube. The solution was swabbed by fully immersing the paper device three times in the solution prior to analysis. In order to ensure each of the paper devices was exposed to the solution for an equal amount of time, each device was immersed in the corresponding solution for ten seconds three times prior to subsequent analysis by wavelength modulated Raman spectroscopy. This ensured that each device was fully covered by the immersion solution.

FIG. 2 shows a system for testing analytes in accordance with the invention. This has an excitation laser that is operable to provide excitation radiation at a range of different wavelengths, a paper microfluidic device for holding a sample in fluid form and a spectrometer for analyzing radiation collected in response to excitation by the laser radiation. The system has a sample holder (not shown) for holding the paper microfluidic device to which an analyte has been applied; an excitation source for exciting Raman scattering in the analyte in the paper microfluidic at a series of different wavelengths, and a detector/spectrometer for capturing a signal from the device at each wavelength. Once the signals are captured, they are analyzed at each wavelength to identify a Raman signal, and use the Raman signal to detect or identify the analyte. This analysis is typically done in the spectrometer or in a computer, for example a standard PC adapted to do the calculations.

The invention has been demonstrated experimentally. For these experiments modulated Raman spectra were acquired using a system based upon a tunable Littman geometry diode laser (Sacher Lasertechnik, centre wavelength of at λ=785 nm, maximum power 1 W, total tuning range 200 GHz). Laser tuning was controlled with a waveform/function generator (Keithley, 50 MHz) that modulated the wavelength. A telescope enlarged the size of the laser beam to fill the back aperture of a microscope objective (Olympus, magnification 40×/NA=0.74) subsequent to passage through a line filter. The inelastically scattered Raman photons were collected through the same objective and coupled through a F/# matcher to a spectrometer with a 400 lines/mm grating. Detection was performed with a deep depletion, back illuminated and thermo-electrically cooled CCD camera (Newton, Andor Technology). Uniform illumination of the sample was realized with a standard Kohler illumination set-up in transmission mode.

The optimization of wavelength modulated Raman spectroscopy has previously been discussed by Mazilu et al, see Praveen B B et al (2012) “Fluorescence suppression using wavelength modulated Raman spectroscopy in fibre-probe-based tissue analysis”, Journal of Biomedical Optics 17:077006; Praveen B B et al (2013) Optimization of Wavelength Modulated Raman Spectroscopy: Towards High Throughput Cell Screening, PLoS ONE 8: e67211; and Mazilu et al, Optimal algorithm for fluorscence for suppression of modulated Raman spectroscopy, Optics Express 18: 11382-11395. The contents of these three papers are incorporated herein by reference.

The optimal conditions for wavelength modulated Raman spectroscopy required optimization of a number of factors including the modulation amplitude, the time constant used for a single spectral acquisition, the sampling rate across one modulation cycle and the number of modulation cycles which are performed per experiment. The standard Raman spectra of a single unmodified paper device showed a number of Raman bands were present which were assigned to the various stretches and bending modes of C—C and C—H cellulose bands. The most intense band detected occurred at 1089 cm⁻¹. To optimize the wavelength modulated Raman spectroscopy conditions, the signal to noise ratio was calculated using the intensity of this band and the standard deviation of the Raman free region as noise. The signal to noise ratio was monitored as each individual set of conditions was modified.

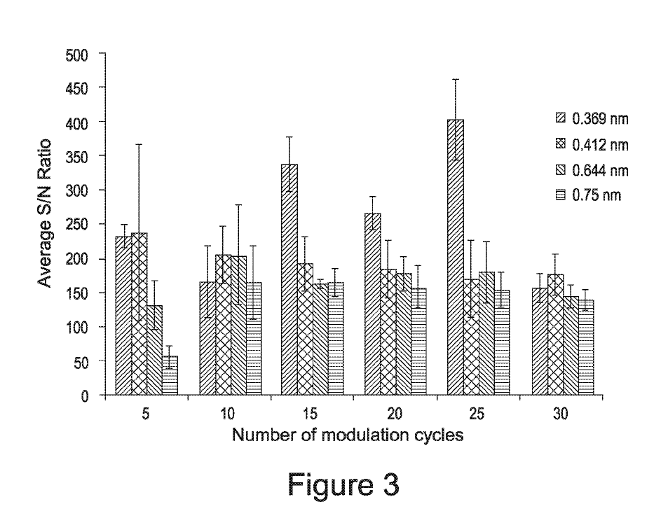

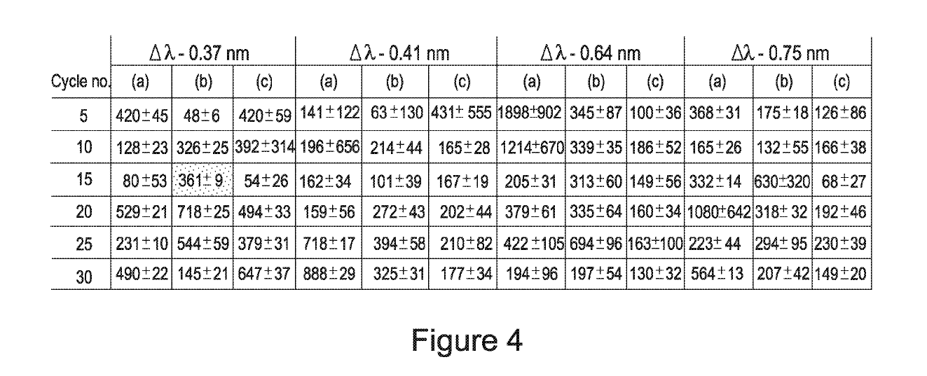

FIG. 3 shows the wavelength modulated Raman spectroscopy measurements of the signal to noise ratio (S/N) of the cellulose band at 1089 cm⁻¹. The bar chart shown represents measurements of the signal to noise ratio S/N using a 4 s exposure time whilst varying the number of kinetic cycles and band-to-band voltage. Error bars shown are the standard deviation of 5 measurements. Measuring changes in the signal to noise ratio S/N based upon the alteration of the various parameters (i.e. modulation amplitude, time constant, sampling rate and number of modulation cycles) highlights a number of factors contribute simultaneously to its optimization. Three different exposure times were tested. The data used is shown in FIG. 4. This shows the measured signal to noise ratio S/N as the number of modulation cycles (5-30) and modulation amplitude (Δλ) were altered for different exposure times: (a) 3 s exposure time, (b) 4 s exposure time and (c) 5 s exposure time. Measurements are an average of 5 replicates.

Based on the optimization experiments, the 4-second exposure time provided the most consistent and highest signal to noise ratio S/N achievable. As the number of modulation cycles was incrementally increased from 5 to 30 cycles, the signal to noise ratio became more consistent, however, this prolonged the time required to perform the analyses. When only five modulation cycles were used significant deviations in the signal to noise ratio were observed. Therefore, a compromise was made to gain a consistent signal to noise ratio S/N over the shortest period and the number of modulation cycles was assessed to be optimum at 15.

Another key variable was the amplitude of the modulation cycle. Four different wavelength modulation amplitudes were explored and each was found to provide an improvement in the signal to noise ratio S/N in comparison to the standard Raman spectrum. The signal to noise ratio S/N obtained for each of the amplitudes tested indicated that no significant enhancement of signal to noise ratio S/N was gained when the amplitude was greater than Δλ=0.37 nm without resulting in increased statistical errors occurring between the measurements performed i.e. increase in the standard deviations of the average signal to noise ratio S/N measurements obtained. Therefore, the highest achievable signal to noise ratio S/N over the shortest number of modulation cycles occurred when using a modulation amplitude of Δλ=0.37 nm, with 15 modulation cycles and 4 s exposure time.

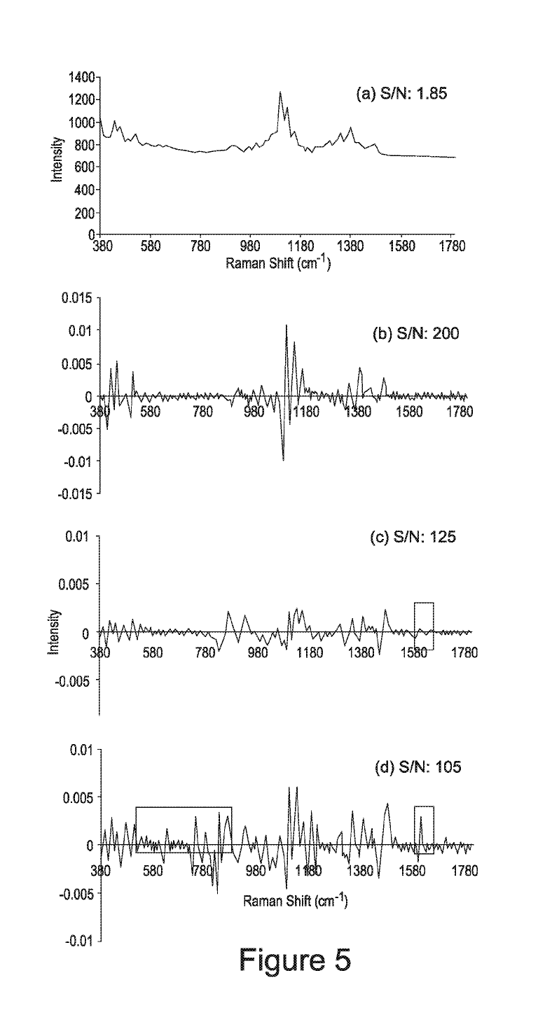

The optimum conditions noted above were implemented for all wavelength modulated Raman spectroscopy experiments discussed below. The significant enhancement of the signal to noise ratio S/N gained from the implementation of these conditions is shown in FIG. 5. This shows spectra of the paper microfluidic device before and after swabbing of pharmaceuticals. In particular, FIG. 5(a) shows a standard Raman spectrum of the paper device; FIG. 5(b) shows a wavelength modulated Raman spectroscopy spectrum of paper only; FIG. 5(c) shows a wavelength modulated Raman spectroscopy spectrum for paper and paracetamol, and FIG. 5(d) shows a wavelength modulated Raman spectroscopy spectrum for paper and ibuprofen. The quoted signal to noise ratios S/N are measured for the 1089 cm ⁻¹ band and are an average of 10 spectra of each individual sample.

As can be seen from FIG. 5(a) and FIG. 5(b), the difference in signal to noise ratio S/N between the standard Raman spectrum of the paper device, and the wavelength modulated Raman spectroscopy spectrum of the paper device is over 100 fold. As well as the increase in the signal to noise ratio S/N, the significant fluorescence background of the paper substrate was removed by using the wavelength modulated Raman spectroscopy. Thus, the distinctive features of the fingerprint spectrum of the paper could easily be identified.

In addition, the wavelength modulated Raman spectroscopy spectra obtained from the blank paper device are easily distinguishable from the Raman spectroscopy spectra obtained from the paracetamol and ibuprofen swabbed samples. As shown in FIG. 5(c) a distinctive band arises at 1600 cm⁻¹, which can be assigned to the amide-stretching band for paracetamol. Distinctive bands can also be detected for the ibuprofen sample in FIG. 5(d) with bands arising between 550 and 800 cm⁻¹, which are distinctive to the ibuprofen spectra. A further band arises at 1590 cm⁻¹, which can be assigned, to the carboxyl group-stretching mode of ibuprofen. This shows that wavelength modulated Raman spectroscopy coupled with paper microfluidics allows the identification of key vibrational bands related to the spectrum of each individual component.

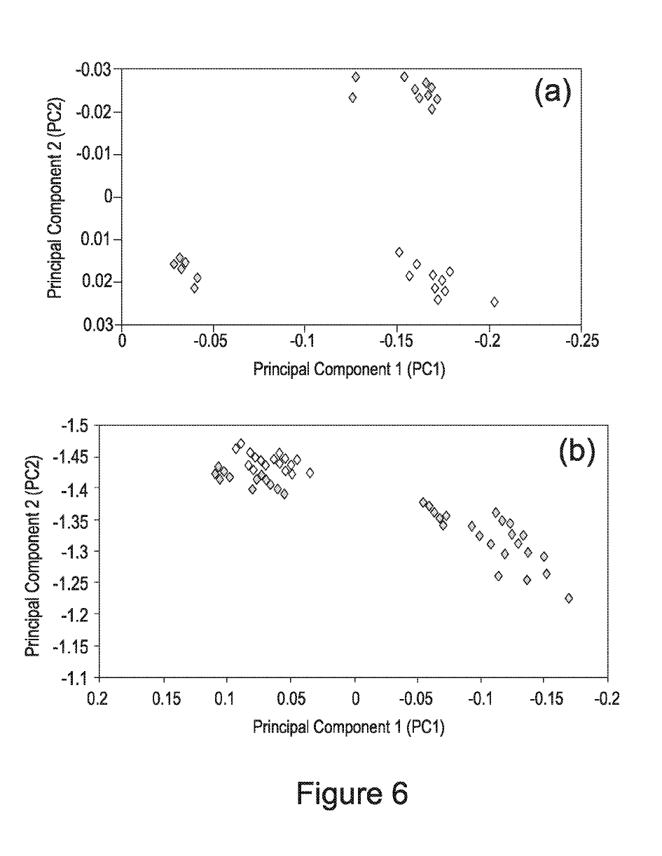

Although the paracetamol spectrum displays an identifiable band difference from the paper substrate and ibuprofen, the differences in spectral position and intensity are minimal. To improve on this, a Principal component analysis was used. In this case, the PCA data set used included two or more of the wavelength modulated Raman spectroscopy spectra from FIG. 5. Performing PCA on these spectra highlights differences in spectral position and intensity. FIGS. 6 to 8 show PCA scatter plots. These Figures show the first two principal components (PC1 and PC2), which demonstrate the greatest variance between samples. However, other higher order components (PC3 and above) could be used. Analysis was performed over multiple spectra of all three types of sample using both standard Raman spectroscopy and wavelength modulated Raman spectroscopy. The resulting data analysis is shown in FIG. 6.

FIG. 6(a) shows a principal component analysis of a wavelength modulated Raman spectroscopy study of paper microfluidics device (green), as well as paper and paracetamol (blue) and paper and ibuprofen (red). In this case, the data set input to the PCA was the spectra of FIGS. 5(b), (c) and (d). FIG. 6(b) shows a standard Raman study of a paper microfluidics device (green), as well as paper and paracetamol (blue) and paper and ibuprofen (red). From FIG. 6(b), it can be seen that the standard Raman spectroscopy analysis results in the production of three data clusters for each of the analytes examined, but the clusters for both the paracetamol and ibuprofen are not adequately separated. Hence, it is not possible to conclusively separate all three components using standard Raman spectroscopy. In comparison, from FIG. 6(a), it can be seen that the use of WMRS and PCA provides a higher discrimination allowing distinctive clusters with adequate separation to be observed, thus ensuring there is no overlap in the spectral analysis. This allows each analyte to be identified.

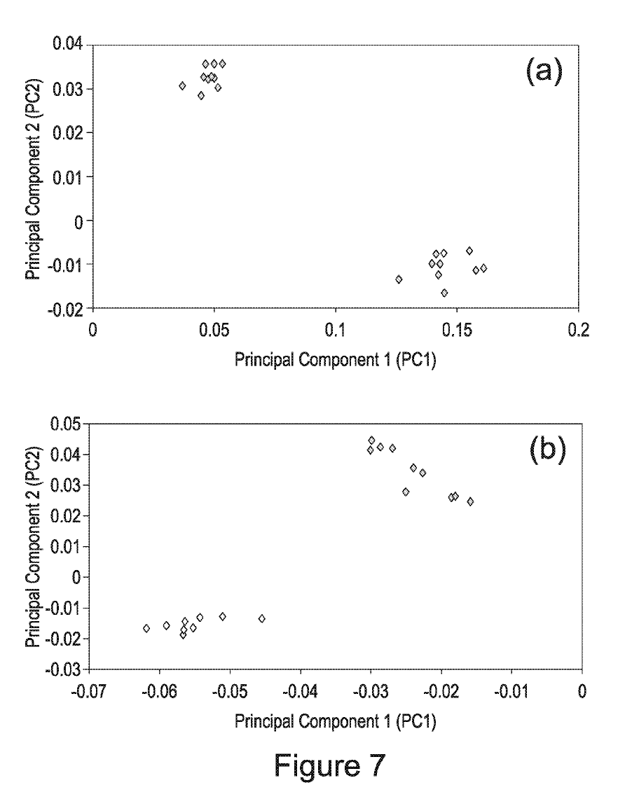

Tests were done to identify the lowest concentration of both paracetamol and ibuprofen detectable on the paper substrate. By serial dilution, a range of concentrations of both paracetamol and ibuprofen were produced. Using the swabbing method and the optimized wavelength modulated Raman spectroscopy conditions noted above, it was possible to achieve adequate cluster separation of both components down-to nanomolar concentrations. The PCA figures showing cluster separation for paracetamol and ibuprofen on the paper substrate are shown in FIGS. 7(a) and (b) respectively. These figures show PCA scatter plots of PC2 vs. PC1 for an analysis of (a) paper and paracetamol (blue) vs. the paper device (green) only and (b) paper and ibuprofen (red) vs. paper device only. FIG. 7 shows that it is possible to separate the data for the pharmaceuticals using the paper microfluidic device qualitatively when concentrations in the nanomolar range were analyzed.

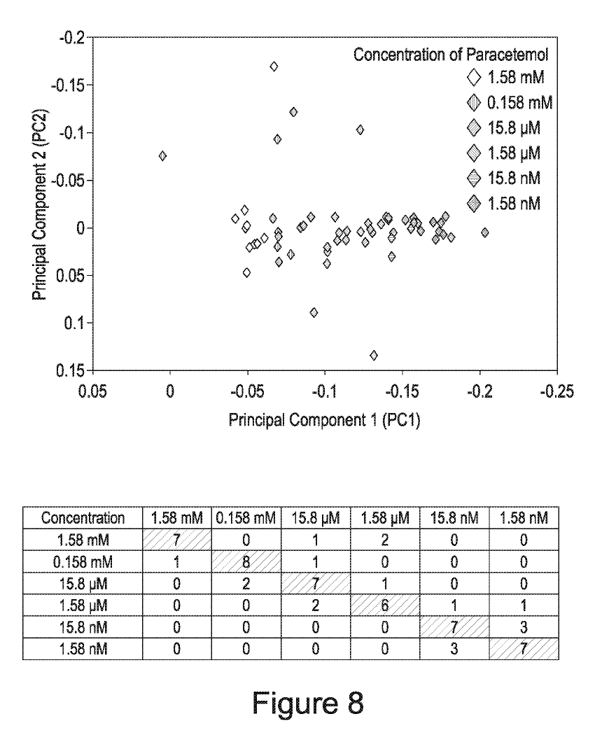

To demonstrate quantitative analysis, a range of concentrations of both paracetamol and ibuprofen were swabbed onto individual paper devices and analyzed by wavelength modulated Raman spectroscopy. The PCA scatter plots and the resulting confusion matrix are shown in FIG. 8, where it can be observed that following the classification experiment it was possible to segregate each individual concentration of paracetamol.

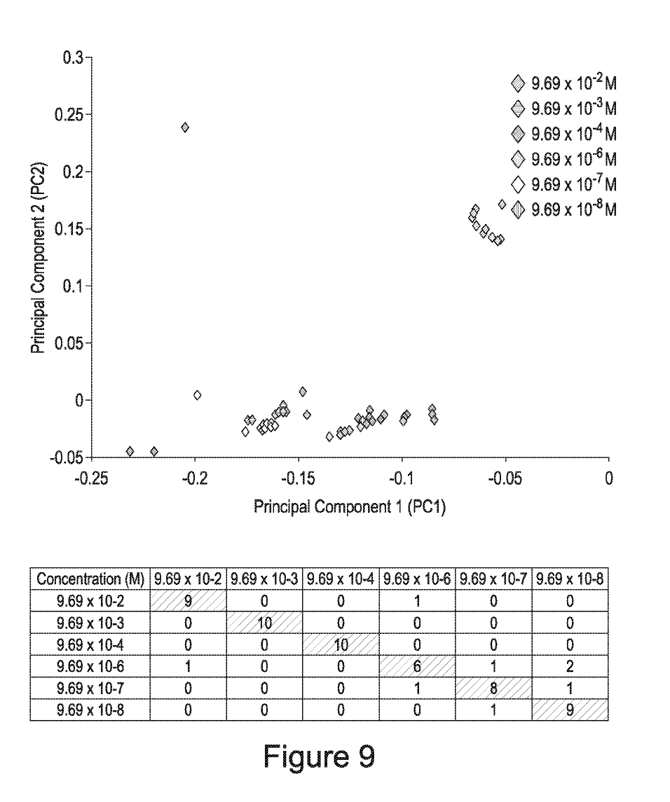

FIG. 8 shows a PCA scatter plot, PC2 vs. PC1, for analysis of varied concentrations of paracetamol on individual paper devices. The table shows a confusion matrix from PCA analysis of a limit of detection study of paracetamol on paper microfluidic devices. Numbers indicate the overlap of data points between each concentration studied. The quantitative identification of individual ibuprofen concentrations was also achieved and this data is found in FIG. 9.

This shows a PCA scatter plot, PC2 vs. PC1, for varied concentrations of ibuprofen on individual paper devices. The table shows the confusion matrix from PCA analysis of a limit of detection study of ibuprofen on paper microfluidic devices. The numbers indicate the overlap of data points between each concentration studied.

The confusion matrix of FIG. 8 employs the “leave one out method”. This method is used to assess the correct classification of an unknown sample after acquiring a set of known samples. More precisely, if N spectra are measured then one random spectrum is chosen to be left out and the remaining (N−1) spectra are used for the PCA. These (N−1) spectra constitute the training set and define a principal component representation of the data where only the first most relevant components are used for the representation. Within this representation, each different analyze will be seen as a small cluster of points. The spectrum that was left out is then projected in the principal component space defined by the training set and classified using the distance to the different clusters. Using this approach, each time with a left out data set, enables the definition of the confusion matrix which is a tally of the correct and incorrect classifications.

In FIG. 8, the diagonal of the matrix represents the number of diluted samples that can be correctly identified and attributed to their correct concentration. Ideally this number would be 10 to represent the fact that the 10 replicate analyses for each concentration had produced data with little inherent variance. However, although this number is not obtained, the confusion matrix shows that the majority of the data clusters together correctly without any significant variance being present. The matrix also highlights that there remains a challenge to improve upon the data acquired with greater variance being generated, as the concentration of paracetamol is sequentially decreased.

The invention may be used in a number of different ways. For example, the invention may be used to detect counterfeit drugs. In this case, known authentic drugs would be analyzed using the paper microfluidics and wavelength modulated Raman spectroscopy of the invention, and a Raman fingerprint would be stored for each authentic drug. The authentic Raman fingerprints for multiple drugs may be stored in a library/database. To authenticate or identify one or more drugs of unknown origin or suspected counterfeit drugs, a solution of the drug would be applied to a paper microfluidic device and tested using wavelength modulated Raman spectroscopy. Ideally, the same concentration of drug and the same wavelength modulation should be used for the test of the counterfeit drug as was used to determine the Raman fingerprint for the authentic drug. Once the Raman fingerprint for the drug of unknown origin or suspected counterfeit drug has been obtained, it is then compared with the Raman fingerprint for the authentic drug. In the event that the Raman signal associated with the drug, and the stored Raman signal associated with a known, authenticated drug are substantially the same, the drug is identified as being authentic. Otherwise, the drug is identified as being counterfeit.

The step of comparing the Raman fingerprints may be done using a principal component analysis. In this case, the dataset for the PCA would be the Raman fingerprint for the unknown/suspected counterfeit drug and the Raman fingerprint for the authentic drug. Of course, it will be appreciated that other techniques for comparing the fingerprints could be used. For example, any suitable multivariate analysis could be used, such as linear discriminate analysis (LDA) or support vector machine (SVM), as well as PCA.

The present invention uses wavelength modulated Raman spectroscopy in combination with paper microfluidics for real-time detection of analytes. The use of wavelength modulated Raman spectroscopy for this application establishes that the common sensitivity issues which plague conventional detection techniques used with paper microfluidics can be overcome, with sensitivity of analyte detection being achieved in the nanomolar range. As a result, it is possible to determine an experimental limit of detection for paracetamol and ibuprofen at concentrations of 1.58 nM and 96.8 nM respectively, when using a combination of wavelength modulated Raman spectroscopy and paper microfluidics. This level of sensitivity is at least equal with current examples of SERS based paper microfluidic detection, but does not require a prolonged fabrication process and is not hindered by substrate reproducibility.

The present invention can be used for real-time detection of multiple analytes simultaneously. There are multiple methods for doing such multiple analyses. The methods discussed above can all be used to distinguish/classify at the same time multiple analytes. To use PCA for example regions in PC space can be defined (PC1 vs PC2) for pure compounds. An unknown multiple analytes sample would correspond to a point in this PC space and its distance to the different regions corresponds to the concentration of each of the pure compounds of interest. This is called partial least-squares regression.

A skilled person will appreciate that variations of the disclosed arrangements are possible without departing from the scope of the invention. Accordingly the above description of the specific embodiment is made by way of example only and not for the purposes of limitations. It will be clear to the skilled person that minor modifications may be made without significant changes to the operation described.

1. A method for detecting or identifying an analyte, the method comprising:
applying an analyte in fluid to a paper microfluidic device;
exciting Raman scattering in the analyte in the paper microfluidic device at a multiple different wavelengths;
capturing a signal at each wavelength; and
analyzing the captured signal at each wavelength to identify a Raman signal associated with the analyte.

2. A method as claimed in claim 1 wherein analyzing the captured signal involves using a principal component analysis to recover the modulated Raman information.

3. A method as claimed in claim 1 comprising varying the excitation wavelength by a predetermined amount, so that the multiple different wavelengths comprises a series of wavelengths separated by said predetermined amount.

4. A method as claimed in claim 1 comprising using the Raman signal to detect or identify the analyte.

5. A method as claimed in claim 4 comprising applying multiple analytes in a fluid to the paper microfluidic, and using the Raman signal to detect or identify each analyte.

6. A method as claimed in claim 1 comprising applying a known analyte to the paper microfluidic device and using the Raman signal as a fingerprint for that analyte.

7. A method as claimed in claim 6 comprising creating a library of at least one fingerprint for at least one known analyte.

8. A method as claimed in claim 6 wherein the known analyte is an authentic drug.

9. A method as claimed in claim 6 comprising comparing the identified Raman signal associated with an unknown analyte with the at least one fingerprint for the at least one known analyte.

10. A system adapted to detecting or identifying an analyte, the system comprising:
a sample holder for holding a paper microfluidic device to which an analyte has been applied;
an excitation source for exciting Raman scattering in the analyte in paper microfluidic at a series of different wavelengths;
a detector for capturing a signal from the device at each wavelength; and
an analyzer for analyzing the captured signal at each wavelength to identify a Raman signal, and use the Raman signal to detect or identify the analyte.

11. A method for detecting counterfeit drugs, the method comprising:
applying drug in fluid to a paper microfluidic device;
exciting Raman scattering in the drug in the paper microfluidic device at a multiple different wavelengths;
capturing a signal at each wavelength;
analyzing the captured signal at each wavelength to identify a Raman signal associated with the drug; and
comparing the Raman signal associated with the drug with a stored Raman signal associated with a known, authenticated drug.

12. A method as claimed in claim 11 wherein the multiple different wavelengths comprise a series of wavelengths separated by the same wavelength difference.

13. A method as claimed in claim 11 wherein the difference between each wavelength in the series is less than 2 nm.

14. A system for detecting counterfeit drugs using a paper microfluidic device, the system being adapted to:
excite Raman scattering in a drug in fluid form applied to the paper microfluidic device at a multiple different wavelengths;
capture a signal at each wavelength;
analyze the captured signal at each wavelength to identify a Raman signal associated with the drug; and
compare the Raman signal associated with the drug with a stored Raman signal associated with a known, authenticated drug.

15. A method as claimed in claim 1, wherein the fluid is a drug.

16. A method as claimed in claim 1 comprising varying the excitation wavelength by 1 nm, so that the multiple different wavelengths comprises a series of wavelengths separated by 1 nm.

17. A method as claimed in claim 11 wherein the difference between each wavelength in the series is 1 nm or less.
